(19) United States
(12) Patent Application Publication
(10) Pub. No.: US 2010/0168805 A1
(43) Pub. Date: Jul. 1, 2010

(54) Oral Prosthesis System Including an Electrostimulation Device Associated with a Wireless Transmission-Reception Device

(75) Inventors: Bruno Colin, Saint Series (FR); Yohan Payan, Allevard (FR)

Correspondence Address: Howard IP Law Group, P.O. Box 226, Fort Washington, PA, 19034, US

(73) Assignee: UNIVERSITE JOSEPH FOURIER, Grenoble (FR)

(21) Appl. No.: 12/594,689

(22) PCT Filed: Apr. 4, 2008
(86) PCT No.: PCT/FR2008/050605

§ 371 Date: Mar. 5, 2010

(30) Foreign Application Priority Data
Apr. 5, 2007 (FR) 0754295

(51) Int. Cl. A61N 1/36; A61N 1/08

(52) U.S. Cl. 607/2

(57) ABSTRACT

An oral prosthesis system comprising an electrical tongue stimulation unit associated with a wireless transceiver device and comprising two microcontrollers, the first microcontroller dedicated to transmit and receive operations, and the second microcontroller dedicated to the control of an electrical stimulation array.

### FIELD OF THE INVENTION

The present invention relates to the forming of electrical tongue stimulation units.

### DISCUSSION OF PRIOR ART

Electrical stimulation units comprise an array of electrodes placed in an individual s hard palate above the tongue in the form of a removable oral prosthesis likely to be attached to the patient s dentition. Such a unit is for example described in U.S. Pat. No. 6,430,450.

The use of electrical tongue stimulation units has been suggested for many applications. Such applications especially aim at increasing a permanently or temporarily disabled person s autonomy by compensating for a temporary or permanent disability. Such units are especially capable of providing the patient with information that his or her body does not give him or her. These applications comprise a rehabilitation after a trauma of a joint or of a body segment due to accidental causes, to a disease, or to surgery.

Such applications are made possible since the patient s tongue comes at regular intervals—by reflex movement—into contact with the array attached to the palate, which enables to provide information to the patient. For example, as described in U.S. Pat. No. 6,430,450, a visual impairment is compensated by the provision of visual information from a camera, linked to the patient to the electrical tongue stimulation unit.

Similarly, it has been suggested to use the electrical tongue stimulation unit to enable the patient to interact with his or her environment. Thus, the patient could control the triggering of an alarm or the displacements of motor-driven equipment such as a bed or a wheelchair, or he or she could control home equipment such as a light, a telephone, a door, or a computer

It has also been suggested for a same electrical tongue stimulation unit to be used to both provide information from the outside to the patient and enable him or her to interact with his or her environment. Thus, U.S. Pat. No. 6,430,450 provides for the patient to be able to start an actuator such as a robotic hand based on the visual data transferred onto his or her tongue.

It has further been provided for the information transfer operations to be performed wirelessly by a wireless transceiver device built in the prosthesis comprising the electrical tongue stimulation unit. Thus, U.S. Pat. No. 6,430,450 provides for the information originating from the camera to be provided to the electrical stimulation unit by wireless transmission/reception.

All these solutions have been provided while there existed no electrical tongue stimulation unit with a wireless transceiver function and were based on the assumption that such devices would be easy to build.

In practice, the building of such devices comes up against many challenges and constraints. Among the latter, the need for a relatively high autonomy, on the order of at least one day of use, should be noted. Another constraint is the bulk. It must be small so that the transceiver device can be contained in the same oral prosthesis as the electrical tongue stimulation unit. Another constraint which combines with the constraint of a small bulk is that the device must have a very low heat dissipation. It must indeed be avoided for the patient s hard palate, tongue, gums, and/or teeth to be exposed to heat for a long time.

An embodiment of the present invention aims at providing an oral prosthesis system comprising an electrical tongue stimulation unit associated with a wireless transceiver.

An embodiment of the present invention aims at providing such a system likely to operate with a low power consumption.

An embodiment of the present invention aims at providing such a system which is of small bulk.

An embodiment of the present invention aims at providing such a system which operates in radio frequency mode.

### SUMMARY OF THE INVENTION

To achieve all or part of these and other objects, an embodiment provides an oral prosthesis system comprising an electrical tongue stimulation unit associated with a wireless transceiver device and comprising two microcontrollers, a first microcontroller dedicated to transmit and receive operations, and a second microcontroller dedicated to the control of an electrical stimulation array.

According to an embodiment, the microcontrollers are connected by a serial interface of universal asynchronous receiver-transmitter type.

According to an embodiment, the microcontrollers are microcontrollers capable of switching from a standby mode to an active mode within a time shorter than 5 μs.

According to an embodiment, the microcontrollers are microcontrollers of very low average current during a standby phase comprising very short periods of periodic activation of the receive device.

According to an embodiment, the average current of the microcontrollers is lower than 15 μA during the standby phase, the periods of activation of the receive device lasting for at most 1 millisecond and occurring once per second.

The present invention also provides an electrical stimulation method using a system according to any of the foregoing embodiments, each of the microcontrollers being in a standby phase during which it initiates periodic controls of occurrence of an interruption until an interruption occurs.

### BRIEF DESCRIPTION OF THE DRAWINGS

The foregoing objects, features, and advantages of the present invention, as well as others, will be discussed in detail in the following non-limiting description of specific embodiments in connection with the accompanying drawings, among which:

FIG. 1 is a diagram schematically illustrating various elements of an oral prosthesis system according to an embodiment of the present invention;

FIG. 2 is a timing diagram illustrating a phase of an operating mode of a system according to an embodiment of the present invention; and

FIGS. 3A and 3B are timing diagrams illustrating another phase of an operating mode of a system according to an embodiment of the present invention.

### DETAILED DESCRIPTION

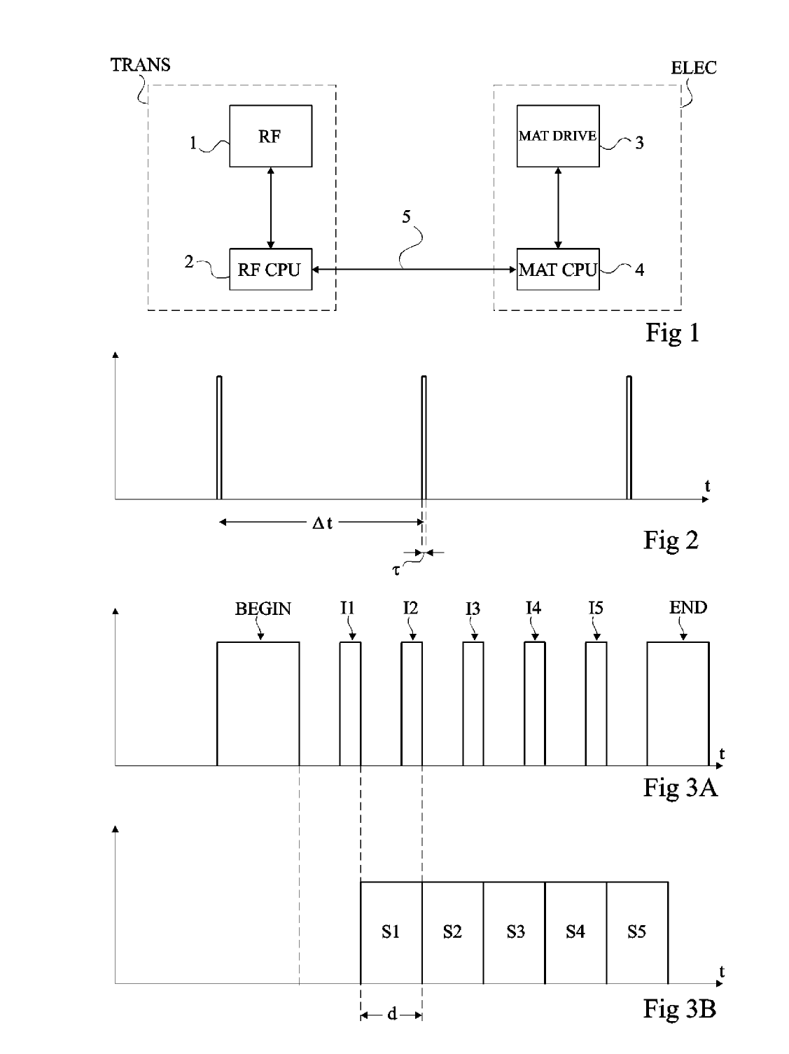

FIG. 1 illustrates a simplified diagram of an oral prosthesis system comprising an electrical tongue stimulation unit associated with a wireless transceiver device according to an embodiment of the present invention.

The system according to an embodiment of the present invention comprises a transceiver block TRANS comprising a radio frequency transceiver RF 1 associated with a processor RFCPU 2 dedicated to transmit/receive operations. The system also comprises an electrical stimulation block comprising an array of electrodes associated with elements for controlling electrical stimulation array MATDRIVE 3 driven by a dedicated processor MATCPU 4. Processors 2 and 4 are associated via a serial interface of universal asynchronous transceiver type (UART) 5.

Surprisingly, using two processors placed in the oral prosthesis provides a lower consumption than the use of a more powerful processor capable of managing transmit/receive operations as well as electrical stimulation device control operations. As will appear from the following description of an embodiment of the system according to an embodiment of the present invention, this is especially due to the fact that each processor may advantageously be placed in low-consumption modes independently from each other.

An operating mode of a system according to an embodiment of the present invention is described hereafter in relation with the simplified timing diagrams of FIGS. 2, 3A, and 3B. FIG. 2 illustrates a standby phase of the system. FIGS. 3A and 3B illustrate an active phase of the system when the system receives data from the outside.

As long as no request of activation of the electrical stimulation array is sent by an external device, the system is in standby mode. During such a mode, transceiver block TRANS becomes periodically active, as illustrated in FIG. 2A, for example, every second. During each of the activity phases triggered by processor 2, block TRANS controls whether any activation request has been transmitted by an external device. In the absence of such a request, the activation time of block TRANS is shorter than one millisecond. As long as no request has been detected, the average consumption is very low, for example, lower than 15 microamperes with conventional processors.

FIGS. 3A and 3B illustrate the activity of transmission block TRANS and of electrical stimulation block ELEC during an active phase of the system. As illustrated in FIG. 3A, during a periodic control, separated by one second from the previous control, block TRANS is assumed to have detected an activation frame BEGIN. Processor 2 then switches block TRANS and block ELEC to the active mode via interface 5. Block TRANS then receives a succession of electrical stimulation instructions comprising the location of the electrode to be activated as well as, possibly, instructions relative to the duration and intensity of the stimulation. For example, FIG. 3A illustrates five successive instructions I1, I2, I3, I4, and I5. Each instruction I corresponds to an electrical stimulation S of an electrode in the array. Preferably, as illustrated by the comparison of FIGS. 3A and 3B, the time interval separating the reception of two successive instructions is optimized so that the corresponding electrical stimulations immediately follow one another. Thus, the end of the reception of first instruction I1 is separated from the end of the reception of second instruction I2 by a time interval d, for example on the order of 100 milliseconds, necessary for the processing of instruction I1 by receive processor 2, its transfer to stimulation processor 4, its processing by said processor, and its transfer to the patient in the form of an electrical stimulation S1.

When the last instruction I5 has been transmitted by an external device, said device interrupts all transmissions and receive processor 2 detects the absence of new instructions and sets the system back to standby.

According to a variation, after the transmission of last instruction I5, the external device transmits an end frame END which notifies the processor that the transmission is over.

Preferably, when the transmission is over, before returning to the standby state, block TRANS transmits an acknowledgement message.

The system operation has been described in receive mode hereabove. It should however be understood by those skilled in the art that the operation in transmit mode is symmetrical. Thus, electrical stimulation processor 4 controls at regular intervals during a standby phase whether instructions are coming from the patient via device 3. If, during one of the controls, it detects that the patient wishes to transmit instructions, it activates block TRANS via interface 5.

According to an embodiment, processors 2 and 4 are very low consumption microcontrollers while having very short activation and deactivation times. Thus, the processors must be able to switch from a standby state to an active state in less than 5 μs while having an average current lower than 15 μA, preferably lower than at most 12 μA, during the very short phases of periodic activation of the transceiver block in the standby phase. As discussed, such phases for example occur every second and are as short as one millisecond. For example, the microcontrollers are of type MSP 430 sold by Texas Instruments or an equivalent microcontroller of the same manufacturer or of another manufacturer. Generally, the system according to an embodiment of the present invention may use any type of microcontroller having a low operating voltage, a low consumption in an operating mode comprising the periodic wake-up management, a low consumption in active mode, and the ability to switch from an inactive mode to an active mode within as short a time as possible, preferably shorter than 5 microseconds. Processor 4 for managing the electrical tongue stimulation array must also be a low-bulk processor of very low consumption in standby mode. Preferably, to decrease the operating consumption, the system is regulated by means of a very-low consumption 2.3-V regulator of LDO type (low drop out voltage). Such a regulator enables to ensure a sufficiently large range of operation without performance variations of the radio frequency transceiver system. Indeed, the performance is stable and guaranteed as long as the battery voltage remains between its nominal voltage, on the order of 2.5 volts, and the regulator voltage.

The device for controlling electrical stimulation array comprises in particular, digital-to-analog converters and multiplexers. Preferably, the components are selected so that this system can be made very rapidly inactive and switches to a mode of very low consumption, preferably below one microampere.

Of course, the present invention is likely to have different variations and modifications which will occur to those skilled in the art. In particular, the transmission protocol has only been described in a very simplified way. It will be within the abilities of those skilled in the art to select and adapt a specific protocol according to the desired application. Similarly, it will be within the abilities of those skilled in the art to select the different elements of the oral prosthesis according to the teachings of the present patent application and to the desired application.

1. An oral prosthesis system comprising an electrical tongue stimulation unit associated with a wireless transceiver device and comprising two microcontrollers, the first microcontroller dedicated to transmit and receive operations, and the second microcontroller dedicated to the control of an electrical stimulation array.

2. The system of claim 1, wherein the first and second microcontrollers are connected by a serial interface of universal asynchronous transceiver type.

3. The system of claim 1, wherein the first and second microcontrollers are microcontrollers capable of switching from a standby mode to an active mode within a time shorter than 5 μs.

4. The system of claim 3, wherein the first and second microcontrollers are microcontrollers of very low average current during a standby phase comprising very short periods of periodic activation of the receive device.

5. The system of claim 4, wherein the average current of the first and second microcontrollers is lower than 15 μA during the standby phase, the periods of activation of the receive device lasting for at most 1 millisecond and occurring once per second.

6. An electrical stimulation method using the system of claim 1, wherein each of the first and second microcontrollers is in a standby phase during which it initiates periodic controls of occurrence of an interruption until an interruption occurs.
